(19) United States
(12) Patent Application Publication
(10) Pub. No.: US 2022/0095795 A1
(43) Pub. Date: Mar. 31, 2022

(54) RESTRAINT BRACKET

(71) Applicant: Pineapple Contracts Unlimited, Kent (GB)

(72) Inventor: Ingemar Jonsson, Oxfordshire (GB)

(21) Appl. No.: 17/485,829

(22) Filed: Sep. 27, 2021

(30) Foreign Application Priority Data
Sep. 28, 2020 (GB) GB2015315.1

(51) Int. Cl. A47B 95/02; F16B 12/34

(52) U.S. Cl. CPC A47B 95/02; F16B 12/34

(57) ABSTRACT

A bracket assembly for securing a bracket to a surface of a furniture item. The assembly includes a bracket plate for securement to the furniture item and a bracket. The bracket has a main body extending to a closed handle. The main body is configured to be securable to the plate such that the handle extends outwardly from the furniture item, and to be removable from the plate when the bracket is not required.

### FIELD OF THE INVENTION

This invention relates to a restraint bracket. More especially the invention relates to a restraint bracket securable to a furniture item, and a method of fixing and removing the bracket to and from an item of furniture.

### BACKGROUND TO THE INVENTION

In hospitals and prisons there can sometimes be the need to restrain a patient or prisoner to a piece of furniture, usually a chair or a bed, for their own safety or the safety of others.

Hospital or prison furniture is usually provided with restraint brackets to which the restraining means can be secured.

A problem with the use of restraint brackets is the risk that can pose to patient when restraint is not required and when the patient is left unattended. In such circumstances the patient, who may be suffering from a mental disorder, could use the bracket to fix a ligature to the furniture in an attempt to commit self-harm or suicide.

The present invention seeks to overcome this problem by providing a restraint bracket which is easily connectable and removable from the furniture so that it is only present when required.

### SUMMARY OF THE INVENTION

In one aspect there is provided a bracket assembly for securing a bracket to a surface of a furniture item, the assembly comprising: a bracket plate for securement to the furniture item; a bracket having a main body extending to a closed handle, the main body being configured to be securable to the plate such that the handle extends outwardly from the furniture item, and to be removable from the plate when the bracket is not required, wherein: the bracket plate includes a keyhole recess having a slot and an aperture in which a locking member is located; the bracket includes a catch member, part of which is locatable in use behind the keyhole slot; and the locking member is movable between a first position, wherein the locking member is located entirely within the keyhole aperture, and a second position, wherein part of the locking member extends out of the aperture to secure the bracket against the plate.

Preferably, the catch member comprises a disc-shaped member elevated above the surface by a stem.

Preferably, the keyhole aperture has a larger dimension than the catch member and the keyhole slot has a lesser dimension than the catch member.

In a second aspect there is provided method of securing a bracket to furniture according to the first aspect, the method comprising the steps of: inserting of the catch member into the aperture of the keyhole recess; moving the bracket to a position where the catch member is located behind the slot of the recess; and unlocking the locking member to enable its extension out of the keyhole aperture.

### BRIEF DESCRIPTION OF THE DRAWINGS

The invention will now be described by way of example only with reference to the accompanying drawings in which:

FIG. 1 is a perspective view of the bracket plate of a restraint bracket assembly constructed in accordance with an embodiment of the invention;

FIG. 2 is a perspective view of a restraint bracket secured to the bracket plate of FIG. 1;

FIGS. 3 and 4 illustrate the mechanism by which the restraint bracket is secured to the plate; and

FIG. 5 is a perspective view of the rear surface of the bracket of the assembly.

### DETAILED DESCRIPTION OF THE INVENTION

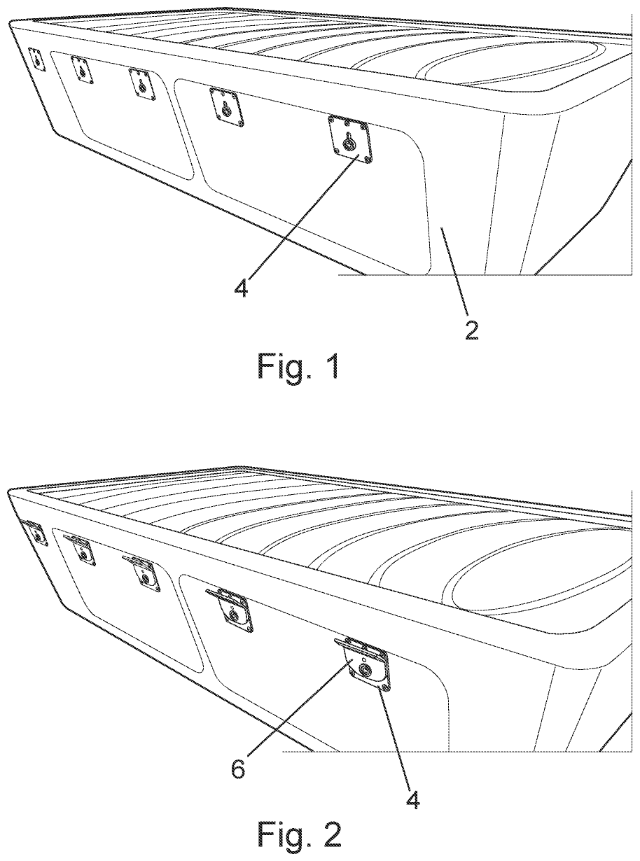

FIGS. 1 and 2 show a restraint bracket assembly secured to a bed 2.

A number of brackets are secured along both side walls of the bed 2 to provide connection points for a restraint to restrain a person flat against the bed 2 when necessary.

An individual bracket assembly will now be described.

The assembly comprises a bracket plate 4 secured to a side surface of the bed 2 and a restraint bracket 6 which can be secured to the plate 4.

FIG. 1 shows the assembly with the restraint bracket 6 removed and FIG. 2 shows the assembly with the bracket secured.

The plate 4 is generally square and made form stainless steel or similar material. The plate 4 is secured to the bed 2 flush with its side wall using standard fixings such as screws or a nut and bolt.

The fixings may be covered or otherwise rendered inaccessible to any unauthorised personnel to prevent the plate from being removed and possibly used as a tool or weapon. The side wall may be recessed to accommodate the plate 4. The plate 4 may alternatively be formed integral with the side wall.

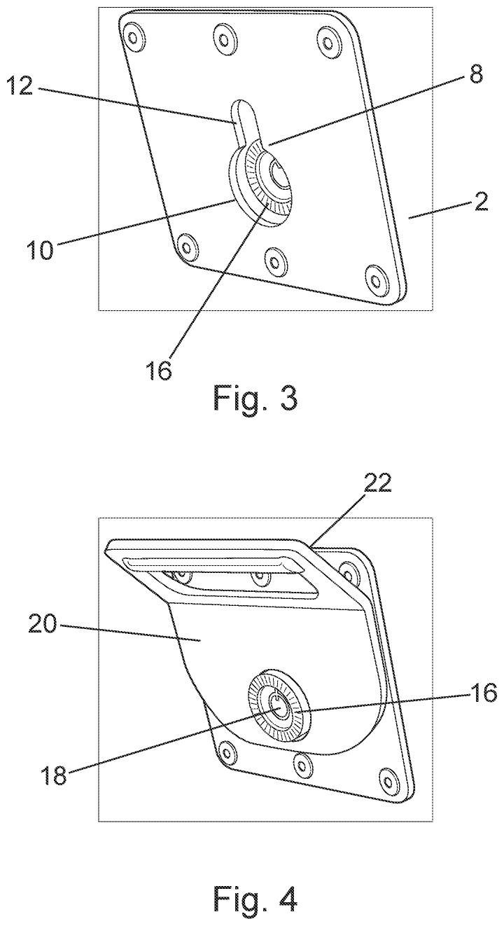

As can be seen in FIG. 3, the plate 4 has a central keyhole recess 8 comprising a generally circular opening 10 and a slotted opening 12. The circular opening 10 of the recess 8 extends to a generally circular bore 14 extending into the plate 4 and in which is located a spring loaded locking member 16. To this end, at least part of the plate 4 has a thickness sufficient to accommodate the locking member 16 to the extent that locking member 16 can be pushed in entirely into the bore 14 as shown in FIG. 3. Alternatively, the plate 4 includes a housing extending from the back of the plate 4 to accommodate the increased depth of the bore 14.

The walls of the bed 2 are preferably hollow to accommodate the depth of the plate 4 (or housing) to maintain the front surface of the plate 4 substantially flush with the outer surface of the bed 2. Alternatively, the wall of the bed 2 has a circular recess into which plate bore 14 can extend.

As can be seen in FIGS. 3 and 4, the locking member 16 is movable between a first position in which it is located within the bore 14 of the recess 8 (FIG. 3) to a second position in which an end portion of the locking member 16 extends out of the recess 8 beyond the front surface of the plate 4 (FIG. 4).

The locking member 16 can be locked in either the first or second position using a key (not shown). To this end, the end surface of the locking member 16 is provided with a keyhole slot 18. Unlocking the member 16 allows it to move within the bore 14.

In order to secure the restraint bracket 6 to the plate 4 and hence to the bed 2, the locking member 16 is first unlocked for movement using the key, and then pushed into the recess 8 to its first position.

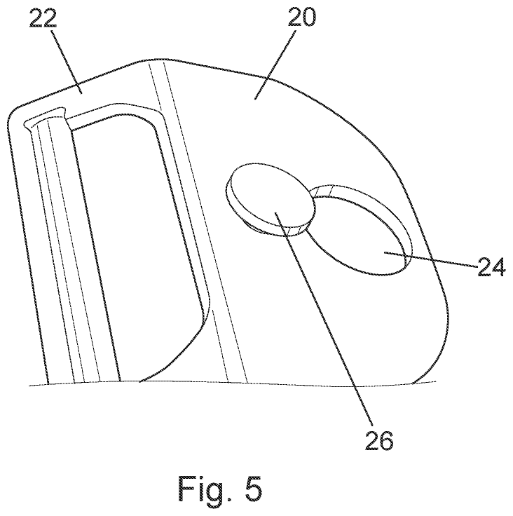

The restraint bracket 6 is preferably made from stainless steel and comprises a generally flat connecting plate section 20 extending to an angled handle section 22. The plate section 20 includes a generally circular aperture 24 (best seen in FIG. 5). The aperture is of substantially the same diameter as the circular opening 10 of the recess 8 to overlap with the opening 10 in use.

As can be seen on FIG. 5, the underside of the plate section 20 includes a disc-shaped catch member 26 elevated above the surface by a central stem (not shown) and oriented to be general parallel with the surface to one side (above in use) of the aperture 26. The diameter of the catch member 26 is less than the diameter of the circular opening 10 of the recess 8 but greater than the width of the slot opening 12 of the recess 8.

To connect the restraint bracket 6 to the plate 4, the locking member 16 is first unlocked using a key to allow the locking member 16 to be pushed into its first position within the recess 8. The locking member 16 is then locked its first position against its spring bias using the key. This allows the plate section 20 of the bracket 6 to be placed against the plate 4 unobstructed.

The catch member 26 is then inserted into the circular opening 10 of the recess 8 at which time the bracket 6 is lifted to guide the stem upwardly into and along the slot opening 12 of the recess 8 with the disc component of the catch member lying behind the slot 12.

Once the bracket 6 is lifted to align the aperture 24 of the bracket 6 with the opening 10 of the recess 8, the locking member 16 is unlocked causing its spring bias to extend out of the recess 8 to its second position wherein an outer portion of the locking member 16 extends through and beyond the bore 14 and the bracket aperture 24. The locking member 16 is locked in its second position using the key.

As can be seen in FIG. 4, the extension of the locking member 16 beyond the surface of the plate 4 and bracket 6 provides an abutment surface to prevent downward movement of the bracket 6 whilst at the same time the catch member 26 prevents the bracket 6 from being away from the plate 4.

The closed handle section 22 of the bracket 6 extends outwardly at a convenient angle from the side of the bed 2 to provide a connection point for a restraint.

When restraint of the patient is no longer required, the restraint bracket 6 can be easily removed from the plate 4 by simply unlocking the locking member 16, pushing it back to its first position to allow downward movement of the bracket 6 thereby releasing the catch member 26 from the slot 12 and out through circular opening 10 of the recess 8.

Finally, the locking member 16 is unlocked to cause the locking member 16 to move back to its second position.

The present invention provides a quick and easy way to secure and remove the bracket 6 to encourage authorised personnel to ensure that there is no feature that could be used as a connection point for a ligature if a suicidal patient is left on the bed unattended.

1. A bracket assembly for securing a bracket to a surface of a furniture item, the assembly comprising:
a bracket plate for securement to the furniture item;
a bracket having a main body extending to a closed handle, the main body being configured to be securable to the plate such that the handle extends outwardly from the furniture item, and to be removable from the plate when the bracket is not required, wherein:
the bracket plate includes a keyhole recess having a slot and an aperture in which a locking member is located;
the bracket includes a catch member, part of which is locatable in use behind the keyhole slot; and
the locking member is movable between a first position, wherein the locking member is located entirely within the keyhole aperture, and a second position, wherein part of the locking member extends out of the aperture to secure the bracket against the plate.

2. A bracket assembly according to claim 1, wherein the catch member comprises is a disc-shaped member elevated above the surface by a stem.

3. A bracket assembly according to claim 2, wherein the aperture of the keyhole recess has a larger dimension than the catch member and the slot of the keyhole recess has a lesser dimension than the catch member.

4. A method of securing a bracket to a furniture according to claim 3, the method comprising the steps of:
inserting of the catch member into the aperture of the keyhole recess;
moving the bracket to a position where the catch member is located behind the slot of the recess; and
unlocking the locking member to enable its extension out of the keyhole aperture.
